SEQUENCE LISTING (4 sequences)

SEQ ID NO: 1 - protein, 13 aa, Artificial sequence
  synthetic sequence
ICVVQDWGHHRCT

SEQ ID NO: 2 - protein, 16 aa, Artificial Sequence
  synthetic sequence
XXCVXQDWGXHRCXXX

SEQ ID NO: 3 - protein, 16 aa, Artificial Sequence
  synthetic sequence
XXCVXQDXGXHRCXXX

SEQ ID NO: 4 - protein, 13 aa, Artificial sequence
  synthetic sequence
ICVWQDWGAHRCT

(19) United States
(12) Patent Application Publication
(10) Pub. No.: US 2011/0008343 A1
(43) Pub. Date: Jan. 13, 2011

(54) Method Of Reducing Tissue Loss In Pancreatic Islet Cell Transplantation

(76) Inventors: John D. Lambris, Bryn Mawr, PA (US); Olie Korsgren, Uppsala (SE); Bo Nillson, Uppsala (SE)

Correspondence Address: POTTER ANDERSON & CORROON LLP;ATTN: JANET E. REED, PH.D., P.O. BOX 951, WILMINGTON, DE, 19899-0951, US

(21) Appl. No.: 12/663,450

(22) PCT Filed: Jun. 6, 2008
(86) PCT No.: PCT/US08/07151

§ 371 Date: Aug. 12, 2010

Related U.S. Application Data
(60) Provisional application No. 60/933,936, filed on Jun. 8, 2007.
(60) Provisional application No. 61/069,047, filed on Mar. 11, 2008.

(51) Int. Cl. A61K 39/395; A61K 38/10; A61P 37/06

(52) U.S. Cl. 424/135.1; 514/21.5; 424/133.1

(57) ABSTRACT

Methods for reducing rejection of pancreatic islet cells transplanted into a subject are disclosed. The methods involve transplanting pancreatic islet cells into a subject in the presence of a complement inhibitor, alone or combined with dextran sulfate.

Pursuant to 35 U.S.C. §202(c), it is acknowledged that the United States government may have certain rights in the invention described herein, which was made in part with funds from the National Institutes of Health under Grant Numbers EB003968, GM62134 and GM069736.

### FIELD OF THE INVENTION

This invention relates to the field of transplantation. Methods for reducing rejection of pancreatic islet cells transplanted into a subject are provided. The methods involve transplanting pancreatic islet cells into a subject in the presence of a complement inhibitor, alone or combined with dextran sulfate.

### BACKGROUND OF THE INVENTION

Various publications, including patents, published applications, technical articles and scholarly articles are cited throughout the specification. Each of these cited publications is incorporated by reference herein, in its entirety.

Clinical islet transplantation is rapidly becoming an established procedure for the treatment of diabetics with uncontrolled hypoglycemia, but the procedure still requires the transplantation of islets from more than one donor to produce insulin independence (Shapiro et al., (2000) N Engl J Med 343:230-238). In recent studies using PET-CT technology in both humans and in pigs, it was demonstrated that less than 50% of the transplanted islets engraft after 2 hours and that the majority of the tissue loss has already occurred during infusion of the islets. This rapid tissue loss is consistent with the observation that the functional capacity of the transplanted islets from up to four donors corresponds to only about 20-30% of that found in a non-diabetic person (Ryan et al., (2001) Diabetes 50:710-719). Together, these reports indicate that only a small fraction of the transplanted islets successfully engraft. The long-term consequences of this marginal engraftment and drastically reduced n-cell mass may explain the observation that a majority of the patients receiving transplants according to the Edmonton protocol become insulin-dependent again within 2-3 years of transplantation (Ryan et al., (2005) Diabetes 54:2060-2069).

The reason for this poor engraftment is complex. A major contributor to the poor outcome of clinical islet transplantation is the occurrence of the destructive instant blood-mediated inflammatory reaction (IBMIR), which leads to loss of transplanted tissue when the islets encounter the blood in the portal vein (Bennet et al., (1995) Diabetes 48:1907-1914; Moberg et al., (2002) Lancet 360:2039-2045). This reaction is triggered by tissue factor (TF) expression by the endocrine cells of the islets, combined with an array of other proinflammatory events, such as the expression of MCP-1 (Piemonti et al., (2002) Diabetes 51:55-65), IL-8, and MIF (Waeber et al., (1997) Proc Natl Acad Sci USA 94:4782-4787; Johansson et al., (2006) Am J Transplantation 6(2):305).

The very rapid destruction of the islets observed in the recent PET/CT studies points to the existence of other damaging reactions in addition to the IBMIR. One component of innate immunity that could cause such rapid destruction is the complement system. The complement system is the first line of immunological defense against foreign pathogens and substances. Its activation through the classical, alternative or lectin pathways leads to the generation of anaphylatoxic peptides C3a and C5a and formation of the C5b-9 membrane attack complex. Complement component C3 plays a central role in activation of all three pathways. Activation of C3 by complement pathway C3 convertases and its subsequent attachment to target surface leads to assembly of the membrane attack complex and ultimately to damage or lysis of the target cells.

Complement activation is a component of the IBMIR, but its activation occurs secondary to the thrombotic reaction (Ozmen et al., (2002) Diabetes 51:1779-1784). The immediate destruction seen by PET/CT suggests that complement is also immediately activated when the islets encounter the blood. Similarly, the involvement of a direct complement attack in allogeneic islet transplantation has been postulated by several investigators (Titus et al., (2003) Transplantation 75:1317-1322). However, no conclusions about the possible involvement of complement in allogeneic islet transplantation have been drawn from these studies, since they were performed using single-cell preparations of pancreatic islets, in which many of the cells become damaged, and they utilized less sensitive immunohistochemical techniques. Furthermore, attempts of others to mitigate destruction of islets exposed to allogenic blood through the use of a complement inhibitor, sCR1, have met with limited success, even though complement was found to be inhibited in those studies (Bennet et al., (1999) Diabetes 48:1907-1914).

The fact that islets derived from more than one donor pancreas still generally required to cure an individual diabetic patient has drawn attention to the limited availability of human islets for transplantation and has sparked interest in the use of islets from alternative sources, particularly the pig (Rood et al., (2006) Cell Transplant. 15: 89). IBMIR is also an obstacle to be surmounted before porcine islets can be used in clinical islet xenotransplantation. IBMIR elicits massive cell destruction when porcine islets are exposed to fresh human blood (Bennet et al., (2000) Transplantation 69: 711). The xenogeneic IBMIR is characterized by activation of platelets and the coagulation and complement systems. This activation is accompanied by infiltration of the islets by polymorphonuclear lymphocytes (PMNs) upon exposure of the islets to whole blood (Bennet et al., (2000) Transplantation 69: 7113).

The occurrence of this deleterious IBMIR is supported by studies demonstrating that porcine islets are immediately destroyed when transplanted intraportally into the liver of non-human primates (Buhler et al., (2002) Xenotransplantation 9: 3; Cantarovich et al., (2002) Xenotransplantation 9: 25.). Kirschof et al. reported that most of their porcine islet xenografts (22-73%) were substantially damaged after 24 h when transplanted into non-immunosuppressed monkeys (Kirchhof et al., (2004) Xenotransplantation 11: 396). The grafts exhibited cell destruction, with deposition of coagulation and complement components and platelets, supporting the contention that the IBMIR contributes to the islet damage in this model. Further support for the importance of the IBMIR comes from the observation that although porcine islets can successfully survive in the liver of diabetic monkeys for more than 100 days (Cardona et al., (2006) Nat. Med. 12: 304; Hering et al., (2006) Nat. Med. 12: 301), very high quantities of islets (25,000 and 50,000 IEQs/kg BW, respectively) are needed to produce normoglycemia in the monkeys, indicating that there is a substantial loss of transplanted tissue.

Thus, there is a need in the art for a method for improving engraftment and reducing rejection of transplanted pancreatic islet cells. This invention addressed those needs.

### SUMMARY OF THE INVENTION

One aspect of the invention features a method for reducing rejection of pancreatic islet cells transplanted into a subject. The method comprises the step of transplanting pancreatic islet cells into a subject in the presence of an effective amount of complement inhibitor, wherein the complement inhibitor inhibits complement activation thereby reducing rejection of the transplanted pancreatic islet cells. Another aspect of the invention features a method for reducing rejection of allogeneic or xenogeneic pancreatic islet cells transplanted into a subject. The method comprises the step of transplanting allogeneic or xenogeneic pancreatic islet cells into a subject in the presence of an effective amount of complement inhibitor and an effective amount of dextran sulfate.

In the methods of the invention, the subject is preferably a human. In some embodiments, the subject has type I diabetes.

In one embodiment, the complement inhibitor inhibits release of C-peptide by the transplanted pancreatic islet cells. In another embodiment, the complement inhibitor inhibits lysis of the transplanted pancreatic islet cells. In yet another embodiment, the transplanted cells have increased engraftment compared to cells transplanted in the absence of the complement inhibitor.

In preferred embodiments, the complement inhibitor is a C3 inhibitor. Examples of C3 inhibitors include compstatin, a compstatin analog, a compstatin peptidomimetic, a compstatin derivative, and combinations thereof.

In other preferred embodiments, the complement inhibitor is an inhibitor of membrane attack complex (MAC) formation or function. Non-limiting examples of such MAC inhibitors include Eculizumab, Pexelizumab and ARC 1905.

In one embodiment, the transplanted pancreatic islet cells are allogeneic. In another embodiment, the islet cells are xenogeneic.

In one embodiment, the dextran sulfate is a low molecular weight dextran sulfate (LMW-DS). In one embodiment, the LMW-DS is about 5000 Da.

In one embodiment, the method results in inhibition of instant blood-mediated inflammatory reaction. In some embodiments, inhibition of instant blood-mediated inflammatory reaction results in inhibition or prevention of at least one of: a decrease in free circulating platelets; infiltration of the transplanted pancreatic islet cells by polymorphonuclear lymphocytes; infiltration of the transplanted pancreatic islet cells by macrophages; infiltration of the transplanted pancreatic islet cells by neutrophils; and an increase in factor XIa-antithrombin (FXIa-AT), factor XIIa-antithrombin (FXIIa-AT), thrombin-antithrombin (TAT) and/or plasmin-alpha 2 antiplasmin (PAP).

Other features and advantages of the invention will be understood from the drawings, detailed description and examples that follow.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A and 1B are a series of graphs and images depicting data for to human islets incubated in AB0-compatible hirudin-treated plasma for 30 minutes. The islets were stained for Clq, C3b/iC3b, C4, C9, IgG, IgM, and MBL. As negative control, an antibody recognizing mouse IgG was used. FIG. 1A is a bar graph of data obtained by COPAS analysis. FIG. 1B is a series of representative images of islets obtained by confocal microscopy (n=5). In non-color copies of the images, FITC staining appears as bright areas, whereas unstained portions appear grey.

FIGS. 2A-2C are a series of graphs depicting data for human islets incubated in AB0-compatible hirudin-treated plasma for 30 minutes in the presence (filled symbols) or absence (open symbols) of compstatin (n=5). FIG. 2A depicts the binding of C3b/iC3b as a percentage of the maximum value. FIG. 2B depicts the generation of C3a (means±SEM) as a function of time. FIG. 2C depicts the generation of sC5b-9 (means±SEM) as a function of time.

FIG. 3 is a series of confocal microscopic images of islets incubated in AB0-compatible hirudin-treated plasma human in the presence (+) or absence (−) of compstatin for 5 and 30 minutes. In non-color copies of the images, PI staining appears as bright areas, whereas unstained portions appear grey.

FIGS. 4A and 4B are graphs depicting data for human islets incubated in AB0 compatible hirudin-treated plasma for 30 minutes in the presence (cross-hatched) or absence (dotted) of compstatin. The islets were stained with PI and analyzed by COPAS, and the presence of C-peptide in the supernatants determined by ELISA.

FIGS. 5A-5C are a series of graphs depicting the correlation between the binding of: IgM and C3b/iC3b (FIG. 5A); IgG and C-peptide (FIG. 5B); and sC5b-9 and C-peptide (FIG. 5C) for pancreatic islet cells incubated in hirudin-treated AB0-compatible plasma.

FIG. 6 is a series of graphs depicting plasma APTT values in transplanted diabetic monkeys (M5-M10) treated with heparin (squares) or LMW-DS (circles) as a function of time post-transplantation.

FIG. 7 is a series of graphs depicting TAT, C3a and C5b-9 quantities in EDTA blood drawn from a femoral vein catheter of transplanted monkeys treated with heparin (squares) or LMW-DS (circles) at varying time points after porcine islet xenotransplantation. TAT, C3a, and C5b-9 are expressed as percentage of the pre-transplant values.

FIG. 8 contains a series of histograms (FIG. 8A) showing how engraftment of islet grafts are scored, and a table (FIG. 8B) showing semi-quantitative scoring of the representative examples shown in the histograms.

FIG. 9 is a series of images depicting immunohistochemical staining of porcine islet grafts and a table of semiquantitative quantification of various components. Porcine islet grafts were retrieved from the liver 24 hours after intraportal xenotransplantation of diabetic monkeys treated with LMW-DS or heparin. The images depict representative expression of insulin and distribution of macrophages, T cells and platelets. Magnification x200. The table depicts data for CD41 (platelets), C3c, C9 (complement components), neutrophil elastase (neurophilic granulocytes), CD68 (macrophages), MAC 387 (macrophages), CD56 (NK-cells), CD3 (T-cells), CD20 (B-cells), IgG, and IgM.

FIGS. 10A-10C are a series of graphs and images related to data for porcine islets incubated in hirudin-treated plasma for 30 minutes. The islets were stained for Clq (n=3); C3b/iC3b (n=5); C4 (n=5); C9 (n=3); IgG (n=5); IgM (n=5); and mannose-binding lectin (MBL; n=3). As a negative control, an antibody recognizing mouse IgG was used (n=5). FIG. 10A depicts large particle flow cytometer data. FIG. 10B depicts confocal microscopy images. FIG. 10C is a graph of the deposition of C3b/iC3b on the islet in the absence and presence of compstatin after analysis by large particle flow cytometry (n=5) (*=p<0.5; **=p<0.01). MFI is mean fluorescent intensity.

FIG. 11 is a graph depicting binding of C3b/iC3b to the surface of microtiter wells containing LMW-DS, after incubation with 10% serum in the presence of increasing doses of compstatin for 30 min at 37° C. Symbol (mg/L LMW-DS was present in the wells): Cross (O); Triangle (10); Square (100); and Diamond (1000).

### DETAILED DESCRIPTION OF ILLUSTRATIVE EMBODIMENTS

One aspect of the invention provides a method for reducing rejection of pancreatic islet cells transplanted into a subject. Specifically, the method comprises transplanting pancreatic islet cells into a subject in the presence of a complement inhibitor, as described in greater detail below.

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques or modifications thereof, are used for chemical syntheses and chemical analyses.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, a “complement inhibitor” is a molecule that prevents or reduces activation and/or propagation of the complement cascade. A complement inhibitor can operate on one or more of the complement pathways, i.e., classical, alternative or lectin pathway.

As used herein, a “C3 inhibitor” is a molecule that prevents or significantly reduces the cleavage of C3 into C3a and C3b.

As used herein, a “MAC inhibitor” is a molecule that prevents the formation and/or function of the membrane attack complex (MAC).

As used herein, a “C5aR inhibitor” is a molecule that prevents or significantly reduces the binding of C5a to the C5a receptor.

As used herein, a “Factor D inhibitor” is a molecule that prevents or significantly reduces the activity of Factor D.

As used herein, “autologous” refers to a biological material derived from the same individual into whom the material will later be re-introduced.

As used herein, “allogeneic” refers to a biological material derived from a genetically different individual of the same species as the individual into whom the material will be introduced.

As used herein, “xenogeneic” refers to a biological material derived from a species different from that of the individual into whom the material will be introduced.

As used herein, a “graft” refers to a cell, tissue or organ that is implanted into an individual, typically to replace, correct or otherwise overcome a defect. The tissue or organ may consist of cells that originate from the same individual; this graft is referred to herein by the following interchangeable terms: “autograft,” “autologous transplant,” and “autologous graft.” A graft comprising cells from a genetically different individual of the same species is referred to herein by the following interchangeable terms: “allograft,” “allogeneic transplant,” and “allogeneic graft.” A graft from an individual to his identical twin is referred to herein as an “isograft,” a “syngeneic transplant,” or a “syngeneic graft.” A “xenograft,” “xenogeneic transplant,” or “xenogeneic implant” refers to a graft from one individual to another of a different species.

As used herein, to “alleviate” a disease, defect, disorder or condition means reducing the severity of one or more symptoms of the disease, defect, disorder or condition.

As used herein, to “treat” means reducing the frequency with which symptoms of a disease, defect, disorder, or adverse condition, and the like, are experienced by a patient.

A “prophylactic” treatment is a treatment administered to a subject who does not exhibit signs of a disease or exhibits only early signs of the disease for the purpose of decreasing the risk of developing pathology associated with the disease.

As used herein, a “therapeutically effective amount” or an “effective amount” is the amount of a composition sufficient to provide a beneficial effect to the individual to whom the composition is administered. If the term “effective amount” or “therapeutically effective amount” is not explicitly stated herein, it is implied that the composition is administered in an effective amount.

As used herein, “insulin dependence” refers to an individual's clinical requirement for exogenous insulin to achieve normal glucose tolerance.

As used herein, “rejection” in the context of transplantation refers to the attack by the immune system of the recipient of a graft on the grafted cell, tissue or organ. Rejection may involve the innate immune system, the adaptive immune system, or both.

As used herein, “reducing rejection” is a relative term and refers to a comparison of rejection in the presence and absence of a therapeutic agent. Reducing rejection encompasses reducing the extent fo rejection, the rate of rejection, the frequency and/or intensity of transplant side effects caused by rejection and combinations thereof. Rejection is assessed by clinical measures standard in the art.

As used herein, “pancreatic islet cells” refers to the clusters of cells in the pancreas called the islets of Langerhans. Islets are made up of several types of cells, including beta cells that make insulin.

As used herein, “instant blood-mediated inflammatory reaction (IBMIR)” refers to one or more of the biological reactions induced in a mammal upon exposure to xenogeneic tissue. IBMIR is generally characterized by activation of platelets and the coagulation and complement systems. Accordingly, biological reactions associated with IBMIR include infiltration of the xenogeneic material by polymorphonuclear lymphocytes (PMNs), macrophages and/or neutrophils; formation of fibrin clots around allogeneic or xenogeneic cells, increases in factor XIa-antithrombin (FXIa-AT), factor XIIa-antithrombin (FXIIa-AT), thrombin-antithrombin (TAT) and/or plasmin-alpha 2 antiplasmin (PAP), and decrease in free circulating platelets (platelet consumption).

As used herein, “low molecular weight dextran sulfate” (LMW-DS) refers to dextran sulfate having an average molecular weight of below 20,000 Da.

As used herein, “high molecule weight dextran sulfate” (HMW-DS) refers to dextran sulfate having an average molecular weight greater than about 500,000 Da.

It is understood that any and all whole or partial integers between any ranges set forth herein are included herein.

### DESCRIPTION

The invention springs in part from the inventors' clear demonstration that complement activation occurs essentially immediately upon exposure of pancreatic islet cells to AB0-compatible plasma. Specifically, complement fragments and immunoglobulins are observed to be deposited on the surface of islets. Deposition of C3b/iC3b is observed within 5 minutes of the start of incubation in AB0-compatible plasma. The presence of a complement inhibitor abolishes deposition of C3b/iC3b on the surface of islets and abrogates generation of fluid-phase complement activation product, as well as inhibiting cell lysis. Notably, and in contrast to previous reports, inhibition of complement resulted in a significant diminution of islet cell destruction upon exposure to allogeneic blood.

The invention also springs in part from the inventors' discovery that a residual complement activation is observed despite inhibition by dextran sulfate of instant blood-mediated inflammatory response in a xenogeneic transplant. The inventors further discovered addition of a complement inhibitor to dextran sulfate treatment inhibits the residual complement activation.

Accordingly, the invention provides a method of reducing immune rejection of pancreatic islet cells transplanted into a subject. The method comprises transplanting pancreatic islet cells into a subject in the presence of a therapeutically effective amount of a complement inhibitor. In particular, the complement inhibitor is selected for its ability to inhibit complement activation from C3 and through the formation of membrane attack complexes (MACs). Advantageously, the method may be used as a prophylactic treatment. By reducing or eliminating the rejection of transplanted pancreatic islet cells that occurs during and immediately after the infusion of islets cells into the a subject, the quantity of transplanted pancreatic islet cells necessary for successful engraftment is expected to be reduced by at least 2-fold, preferably 3-fold and more preferably 4-fold, compared to the quantity necessary in the absence of a complement inhibitor. Furthermore, the method is contemplated to prolong the function of the engrafted pancreatic islet cells, compared to pancreatic islet cells transplanted in the absence of a complement inhibitor. In addition, the method is contemplated to prolong the reduction or elimination of insulin dependency resulting from transplantation of pancreatic islet cells when practiced with an insulin dependent subject.

In one embodiment, the invention further comprises transplanting pancreatic islet cells into a subject in the presence of a therapeutically effective amount of a complement inhibitor and a therapeutically effective amount of dextran sulfate. This method is particularly advantageous in reducing graft rejection in allogeneic or xenogeneic transplantation. Without wishing to be bound by theory, it is believed that dextran sulfate inhibits one or more aspects of instant blood-mediated inflammatory reaction and complement inhibitor inhibits complement activation, which together serves to reduces destruction of the transplanted cells and thus reduces rejection.

The method of the invention may be practiced with any vertebrate having a complement system, including but not limited to, non-mammals and mammals. Mammals include humans, non-human primates, goats, sheep, horses, mice, rats and the like. Preferably, the method is practiced with a human recipient of a pancreatic islet cell graft.

The method may be practiced in the transplant of any pancreatic islet cells for any reason. Typically, pancreatic islet cells are transplanted into subjects with type 1 diabetes in order to reduce or eliminate insulin dependency and to reduce the frequency and/or severity of episodes of hypoglycemia. However, the invention should not be construed as limited to use in subjects with type 1 diabetes.

Pancreatic islet cells for use in the method are obtained using conventional methods known in the art. The cells may be from the same species or a different species as the recipient subject. Thus, in one embodiment, the method is practiced in an allogeneic transplantion of pancreatic islet cells. In another embodiment, the method is practiced in a xenogeneic transplantion. The pancreatic islet cells for transplant may be obtained from a cadaver, a living donor, or a combination thereof. In addition, pancreatic islet cells obtained by differentiation of stem cells, embryonic or adult, may also be used in the method of the invention. The pancreatic islet cells may be genetically modified, for instance, to monitor engraftment, viability or other property of the transplanted cells, or to provide an additional therapeutic benefit by the expression of an exogenous coding sequence or modulation of an endogenous coding sequence.

A single complement inhibitor may be administered or two or more different complement inhibitors may be administered in the practice of the method of the invention. In one method of the invention, the method comprises administration of only a complement inhibitor. In other embodiments, other biologically active agents are administered in addition to the complement inhibitor in the method of the invention. Non-limiting examples of other biologically active agents useful in the invention include immunosuppressive agents, such as daclizumab, sirolimus, rapamycin derivatives (e.g., SDZ RAD), cyclosporin and tacrolimus. Preferably, however, the method excludes the administration of a biologically active agent that is a 2-aminopropane-1,3-diol compound, such as 2-amino-2-[2-(4-octylphenyl)ethyl]-1,3-diol (FTY720).

Any complement inhibitor, as defined herein, may be used in the method of the invention. Preferably, the complement inhibitor is one that inhibits the cleavage of C3 to C3a and C3b, or is one that targets downstream events leading to the formation of membrane attack complexes (MACs). MACs are believed to be important in islet cell rejection. Complement inhibitors include, but are not limited to, C3 inhibitors, MAC inhibitors, Factor D inhibitors and C5aR inhibitors. Inhibitors may act directly or indirectly. For instance, Eculizumab (Alexion Pharmaceuticals, Cheshire, Conn.), an anti-05 antibody, inhibits MAC formation by binding to C5 and preventing its cleavage into C5a and C5b; C5b plays a critical role in initiating assembly of MACs. Pexelizumab, an scFv fragment of Eculizumab, has the same activity. Similarly, ARC1905 (Archemix), an anti-C5 aptamer, binds to and inhibits cleavage of C5, inhibiting the generation of C5b and C5a, and thus inhibiting MAC formation. Factor D is inhibited by diisopropyl fluorophosphate. TNX-234 (Tanox) binds to and inhibits Factor D. Other complement inhibitors include antibodies against Factor B (e.g., TA 106, Taligen) and against properdin (Novelmed). Antibodies or other agents that inhibit binding of C3b/iC3b to the surface of pancreatic islet cells are also contemplated in the method of the invention. See also Morikis et al. (2002, Biochem Soc. 30:1026-1036) and Wetsel et al. (in: Therapeutic Interventions in the Complement System, Vol. 9 (Lambris et al., eds) Humana Press, Totowa, N.J., 2000).

To the extent that interaction of C5a anaphylatoxin and its receptor, C5aR, is involved in islet cell rejection, a number of inhibitors are available for reducing or inhibiting that interaction. Acetyl-Phe-[Orn-Pro-D-cyclohexylalanine-Trp-Arg] (AcF[OPdChaWR]; PMX-53; Peptech) is a small cyclic hexapeptide that is a C5aR antagonist. Analogs of PMX-10 (e.g., PMX-201 and PMX-205) that also function as C5aR antagonists are also available (see for instance Proctor et al., 2006, Adv Exp Med. Biol. 586:329-45 and U.S. Pat. Pub. No. 20060217530). Neutrazumab (G2 Therapies) binds to C5aR, thereby inhibiting binding of C5a to C5aR. TNX-558 (Tanox) binds to and neutralizes C5a. Eculizumab indirectly inhibits C5aR by preventing the formation of C5a, the ligand of C5aR. Similarly, ARC1905 also inhibits the generation of C5a by binding to and preventing cleavage of C5.

Preferably, the C3 inhibitor is compstatin or a compstatin analog, derivative or peptidomimetic. Compstatin is a small molecular weight disulfide bonded cyclic peptide having the sequence Ile-Cys-Val-Val-Gln-Asp-Trp-Gly-His-His-Arg-Cys-Thr (SEQ ID NO. 1). Examples of compstatin analogs, derivates and peptidomimetics are described in the art. See, for instance, U.S. Pat. No. 6,319,897, WO/1999/013899, WO/2004/026328, and Morikis et al (1999, “Design, Structure, Function and Application of Compstatin” in Bioactive Peptides in Drug Discovery and Design: Medical Aspects, Matsoukas et al., eds., IOS Press, Amsterdam NL).

An exemplary compstatin analog comprises a peptide having a sequence: Xaa1-Cys-Val-Xaa2-Gln-Asp-Trp-Gly-Xaa3-His-Arg-Cys-Xaa4 (SEQ ID NO. 2); wherein:

Xaa1 is Ile, Val, Leu, Ac-Ile, Ac-Val, Ac-Leu or a dipeptide comprising Gly-Ile;

Xaa2 is Trp or a peptidic or non-peptidic analog of Trp;

Xaa3 is His, Ala, Phe or Trp;

Xaa4 is L-Thr, D-Thr, Ile, Val, Gly, or a tripeptide comprising Thr-Ala-Asn, wherein a carboxy terminal —OH of any of the L-Thr, D-Thr, Ile, Val, Gly or Asn optionally is replaced by —NH₂; and the two Cys residues are joined by a disulfide bond. Xaa1 may be acetylated, for instance, Ac-Ile. Xaa2 may be a Trp analog comprising a substituted or unsubstituted aromatic ring component. Non-limiting examples include 2-napthylalanine, 1-naphthylalanine, 2-indanylglycine carboxylic acid, dihydrotryptophan or benzoylphenylalanine. Ac-ICV(1MeW)QDWGAHRCT-NH₂ (SEQ ID NO. 4) is a potent compstatin analog where Xaa1 is Ile, Xaa2 is 1-methyltryptophan, Xaa3 is Ala and Xaa4 is Thr.

Another exemplary compstatin analog comprises a peptide having a sequence: Xaa1-Cys-Val-Xaa2-Gln-Asp-Xaa3-Gly-Xaa4-His-Arg-Cys-Xaa5 (SEQ ID NO. 3); wherein:

Xaa1 is Ile, Val, Leu, Ac-Ile, Ac-Val, Ac-Leu or a dipeptide comprising Gly-Ile;

Xaa2 is Trp or an analog of Trp, wherein the analog of Trp has increased hydrophobic character as compared with Trp, with the proviso that, if Xaa3 is Trp, Xaa2 is the analog of Trp;

Xaa3 is Trp or an analog of Trp comprising a chemical modification to its indole ring wherein the chemical modification increases the hydrogen bond potential of the indole ring;

Xaa4 is His, Ala, Phe or Trp;

Xaa5 is L-Thr, D-Thr, Ile, Val, Gly, a dipeptide comprising Thr-Asn or Thr-Ala, or a tripeptide comprising Thr-Ala-Asn, wherein a carboxy terminal —OH of any of the L-Thr, D-Thr, Ile, Val, Gly or Asn optionally is replaced by —NH₂; and the two Cys residues are joined by a disulfide bond. The analog of Trp of Xaa2 may be a halogenated trpytophan, such as 5-fluoro-1-tryptophan or 6-fluoro-1-tryptophan. The Trp analog at Xaa2 may comprise a lower alkoxy or lower alkyl substituent at the 5 position, e.g., 5-methoxytryptophan or 5-methyltryptophan. In other embodiments, the Trp analog at Xaa2 comprises a lower alkyl or a lower alkenoyl substituent at the 1 position, with exemplary embodiments comprising 1-methyltryptophan or 1-formyltryptophan. In other embodiments, the analog of Trp of Xaa3 is a halogenated tryptophan such as 5-fluoro-1-tryptophan or 6-fluoro-1-tryptophan.

Other C3 inhibitors include vaccinia virus complement control protein (VCP) and antibodies that specifically bind C3 and prevent its cleavage. Anti-C3 antibodies useful in the present invention can be made by the skilled artisan using methods known in the art. See, for instance, Harlow, et al. (1988, In: Antibodies, A Laboratory Manual, Cold Spring Harbor, N.Y.), Tuszynski et al. (1988, Blood, 72:109-115), U.S. patent publication 2003/0224490, Queen et al. (U.S. Pat. No. 6,180,370), Wright et al., (1992, Critical Rev. in Immunol. 12(3,4):125-168), Gu et al. (1997, Thrombosis and Hematocyst 77(4):755-759) and Burton et al., (1994, Adv. Immunol. 57:191-280). Anti-C3 antibodies are also commercially available.

The dextran sulfate, or a pharmaceutically acceptable derivative thereof, useful in the practice of the invention according to the invention may have a molecular weight from low molecular weight dextran sulfate (LMW-DS), e.g. from a few hundred or thousand Daltons (Da), to high molecular weight dextran sulfate (HMW-DS), generally with a molecular weight over 500,000 Da, e.g. >1,000,000 Da. In one embodiment, LMW-DS is used. Without wishing to be bound by theory, the effect of dextran sulfate molecules on IBMIR may be related to sulfur content, i.e., the number of sulfate groups per glucosyl residue in the dextran chain. The average sulfur content for LMW-DS may be about 10 to 25%, such as 15 to 20%, corresponding to about two sulfate groups per glucosyl residue. For dextran sulfate with an average molecular weight higher than 20,000 Da, a larger sulfur content may be employed.

The invention encompasses the use of pharmaceutical compositions of a complement inhibitor and/or dextran sulfate to practice the methods of the invention. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art

As used herein, the term “pharmaceutically-acceptable carrier” means a chemical composition with which a complement inhibitor or dextran sulfate may be combined and which, following the combination, can be used to administer the complement inhibitor or dextran sulfate to a mammal.

As used herein, the term “physiologically acceptable” ester or salt means an ester or salt form of the active ingredient which is compatible with any other ingredients of the pharmaceutical composition, which is not deleterious to the subject to which the composition is to be administered.

The pharmaceutical compositions useful for practicing the invention may be administered to deliver a dose of between 1 ng/kg/day and 100 mg/kg/day. In one embodiment, the invention envisions administration of a dose which results in a concentration of a complement inhibitor between 1 μM and 10 μM in a recipient of transplanted pancreatic islet cells. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The concentration of administered dextran sulfate, or derivatives thereof, according to the present invention should not be too high in order to minimize any side-effects associated with dextran sulfate. The amounts of dextran sulfate, or derivatives thereof, in the formulation will depend on the severity of the condition, and on the patient to be treated, as well as the actual formulation and administration route employed, and may be determined non-inventively by the skilled person. In most clinical situations suitable doses of dextran sulfate, or derivatives thereof, in the therapeutic and/or prophylactic treatment of mammalian, especially human, patients are those that give a mean blood concentration below 5 mg/ml, probably less than 2 mg/ml and especially less than 1 mg/ml. A preferred concentration range is between 0.01 mg/ml and 1 mg/ml dextran sulfate, such as more than 0.05 mg/ml, more than 0.08 mg/ml or more than 0.1 mg/ml and/or less than 0.8 mg/ml, less than 0.6 mg/ml, less than 0.4 mg/ml or less than 0.2 mg/ml, e.g. within the concentration range of 0.01 mg/ml and 0.2 mg/ml and/or 0.05 mg/ml and 0.2 mg/ml. In any event, the physician or the skilled person will be able to determine the actual dosage, which will be most suitable for an individual patient, which may vary with the age, weight and response of the particular patient. The above-identified dosages are examples of preferred dosages of the average case. However, there can be individual instances where higher or lower dosage ranges are merited, and such are within the scope of the invention.

Depending on the form of transplantation, the site of transplantation, and the patient, to be treated, as well as the route of administration, the compositions may be administered at varying doses. The pharmaceutical composition may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, among others.

The pharmaceutical composition comprising a complement inhibitor and/or dextran sulfate may be administered concurrently with the pancreatic islet cells or immediately before or after administration of the pancreatic islet cells. Preferably, the pharmaceutical composition is administered immediately before the administration of the islet cells, during the administration of the islet cells, or both. With regard to dextran sulfate, the dose of dextran sulfate may be administered as a bolus prior to transplantation. In some embodiments, the dose of dextran sulfate is administered partially as a bolus prior to transplantation, followed by the remaining portion administered at the time of transplantation. In some embodiments, the remaining portion is administered at the time of transplantion and thereafter as a continuous administration for several hours to several days. Post-transplantation administration of complement inhibitor and/or dextran sulfate as needed is also contemplated.

Pharmaceutical compositions that are useful in the methods of the invention may be administered systemically in oral solid formulations, parenteral, ophthalmic, suppository, aerosol, topical or other similar formulations. In addition to a complement inhibitor, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer a complement inhibitor according to the methods of the invention.

Dextran sulfate, and derivatives thereof, may be administered orally, intravenously, intraperitoneally, subcutaneously, buccally, rectally, dermally, nasally, tracheally, bronchially, topically, by any other patenteral route or via inhalation, in the form of a pharmaceutical preparation comprising the active ingredient in a pharmaceutically acceptable dosage form.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, in microbubbles for ultrasound-released delivery or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The following examples are provided to describe the invention in greater detail. They are intended to illustrate, not to limit, the invention.

### Example 1

### Materials and Methods

Islets isolation: Islets of Langerhans were isolated using a modification of the previously described semi-automated digestion-filtration method (Moberg et al., 2002, Lancet 360: 2039-2045; Lakey et al., 1999, Cell Transplant 8: 285-292; Ricordi et al., 1988, Diabetes 37: 413-420), followed by purification on a continuous density gradient in a refrigerated COBE 2991 centrifuge (COBE Blood Component Technology, Lakewood, Colo., USA). Islet volume and purity were determined by microscopic sizing on a grid after staining with diphenylthiocarbazone. The purity of the islets ranged from 40 to 80%.

The islet preparations were cultured in CMRL 1066 culture medium (Mediatech, Inc. Herndon, Va., USA) supplemented with 10 mM nicotinamide (Sigma Aldrich, Schnelldorf, Germany), 10 mM HEPES buffer (Invitrogen, Paisley, Scotland), 0.25 μg/mL Fungizone® (Invitrogen), 50 μg/mL gentamycin (Invitrogen), 2 mM L-glutamine (Invitrogen), 10 μg/mL ciprofloxacin (Bayer AG, Leverkusen, Germany) and 10% (v/v) heat-inactivated AB0-compatible human serum. Islet cultures were kept at 37° C. in humidified air containing 5% CO₂. Human islets, cultured for 2-7 days, were collected and washed twice in cold 1× phosphate-buffered saline (PBS).

Coating of test tubes with heparin: Test tubes (2 ml, Ellerman) were coated with the Corline heparin surface (Corline, Uppsala, Sweden) according to the manufacturer's recommendations. The surface concentration of heparin was 0.5 μg/cm², corresponding to approximately 0.1 IU/cm², or an antithrombin binding capacity of 2-4 pmol/cm². The heparinized surface has previously been shown to be stable, with no leakage of heparin detected over a period of 1 hour.

Preparation of human hirudin-treated plasma: Blood was Drawn from Healthy blood donors into 7-ml tubes containing 500 μg of hirudin (Refludan; Pharmion Ltd, Cambridge, UK), a specific inhibitor of thrombin, in order to investigate complement activation in the absence of anticoagulants and activation of the coagulation system. For each experiment, blood was drawn from an individual with blood group compatibility to the islet donor. The plasma was harvested after centrifugation at 3300×g for 15 minutes and thereafter stored at −70° C.

Complex object parametric analyzer and sorter (COPAS) analysis: The fluorescence-stained islets were analyzed using a complex object parametric analyzer and sorter (COPAS; Union Biometrica, Somerville, Mass.), which is a large-particle-based flow cytometry instrument. One thousand islets were collected using a 488/514 multi-line laser, and positive cells were sorted out for further analysis by confocal microscopy. The COPAS flow cytometry data were analyzed using CellQuest™ Pro software (BD Biosciences Immunocytometry Systems). Data are reported as mean fluorescent intensity (MFI).

Confocal microscopy: Stained islets were contained in a drop of PBS in a small Petri dish and protected from light before examination in the confocal microscope (Zeiss 510 Meta confocal, Carl Zeiss, Jena, Germany). Examination of the stained islets was performed using the 488-nm laser at 10× magnification. Counterstaining with 4′,6-diamidino-2-phenylindole (DAPI) was used to visualize the nuclei of living islet cells. For settings using a gray background, DAPI staining was not necessary.

Analysis of islets by COPAS and confocal microscopy: Approximately 5000 islets were added to 200 μl of human AB0-compatible hirudin-plasma or whole blood in heparinized test tubes. In some experiments, AB0-compatible hirudin-treated plasma was pre-incubated with or without 10 μM Ac-ICV(1MeW)QDWGAHRCT-NH₂ (SEQ ID NO: 4; Katragadda et al., (2006) J Med Chem 49:4616-4622), a potent compstatin analog, for 15 minutes at 37° C. before the islets were added. The mixture of islets and plasma was incubated, with gentle shaking, at 37° C. for up to 30 minutes.

After centrifugation and addition of 10 mM EDTA (final concentration), the supernatants were stored at −70° C. The remaining islets were washed twice in cold PBS as described above. In order to visualize necrotic cells, propidium iodide (PI) was added to the islets 5 minutes before COPAS analysis. Islets that had not been incubated in plasma were used as controls. The islets were also examined by confocal microscopy.

FITC-labeled antibodies recognizing the following proteins were diluted and used according to the manufacturer's recommendations: Clq (AbCam, Cambridge, UK), C3c (for detection of C3b and iC3b), C4, C9, mannose-binding lectin (MBL), IgG, and IgM (DakoCytomation, Glostrup, Denmark). Irrelevant mouse IgG (DakoCytomation) was used as a negative control. In order to analyze membrane regulators of complement, antibodies against CD46, CD54, CD55, CD59 (BD Bioscience) IgM, and IgG (DakoCytomation) were used. All of these antibodies were FITC-conjugated except for CD59, which was conjugated with R-phycoerythrin (R-PE). The islets were incubated with each antibody, with rotation on ice, for 30 minutes. After washing, the islets were treated with 1% formaldehyde (Apoteket, Gothenburg, Sweden) and held on ice until analyzed by COPAS and confocal microscopy.

ELISAs: All of the ELISAs used the following solutions. PBS containing 0.05% Tween 20, 1% (w/v) BSA, and 10 mM EDTA was used as the working buffer. PBS containing 0.05 Tween 20 was used as the washing buffer. The color substrate was 1,2-phenylenediamine dihydrochloride in 0.1 M citrate, pH 5.

C3a: Plasma was diluted 1/1000 in working buffer and analyzed as described previously (Enkdahl et al., 1992, Scand J Immunol 35: 35-91). Monoclonal antibody 4SD17.3 was used as the capture antibody. Bound C3a was detected with biotinylated rabbit anti-C3a diluted 1/150, followed by HRP-conjugated streptavidin (Amersham, Little Chalfort, UK), diluted 1/500. Zymosan-activated serum, calibrated against a solution of purified C3a, was used as a standard, and the values are given as ng/ml.

sC5b-9: Plasma was analyzed using a modification of the EIA described by Mollnes et al., 1985, Scand J Immunol 22: 197-202. Plasma diluted 1/5 was added to microtiter plates coated with anti-neoC9 monoclonal antibody MCaE11. sC5b-9 was detected with a polyclonal anti-C5 antibody diluted 1/500 (Dako A/S, Denmark), followed by HRP-conjugated anti-rabbit Ig diluted 1/500 (Dako). Zymosan-activated serum, defined to contain 40,000 arbitrary units (AU), was used as a standard.

C-peptide: Plasma was diluted 1/200 and analyzed using a C-peptide ELISA (Mercodia, Uppsala, Sweden).

Results:

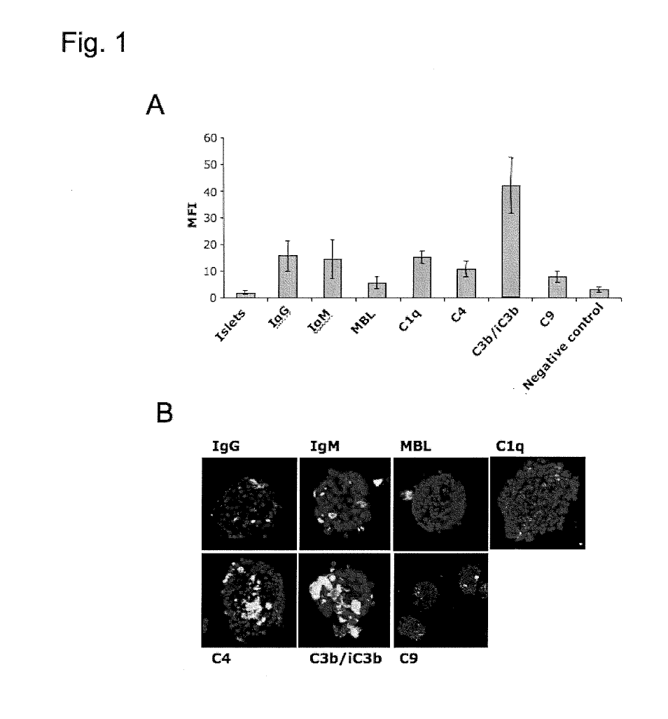

Binding of complement components and expression of complement regulators: To determine whether human islets bind complement proteins after incubation in AB0-compatible hirudin-treated plasma, the islets were stained with FITC-conjugated antibodies recognizing IgG, IgM, Clq, C3b/iC3b fragments, C4, C9, CRP, and MBL. Antibodies against IgG, IgM, Clq, C4, and C3 bound strongly to the islets, but the binding of MBL and C9 was less prominent (FIG. 1A).

Analysis by confocal microscopy revealed that the most extensive binding was seen for antibodies against C4 and C3, with C4 and C3 being found over large areas of the islets, and IgG, IgM, Clq, and C9 being detected in small, discrete spots all over the islet surface (FIG. 1B). No binding of MBL was detected by confocal microscopy.

No expression of the complement regulators MCP (CD46), DAF (CD55), and CD59 was seen on the surface of the islets.

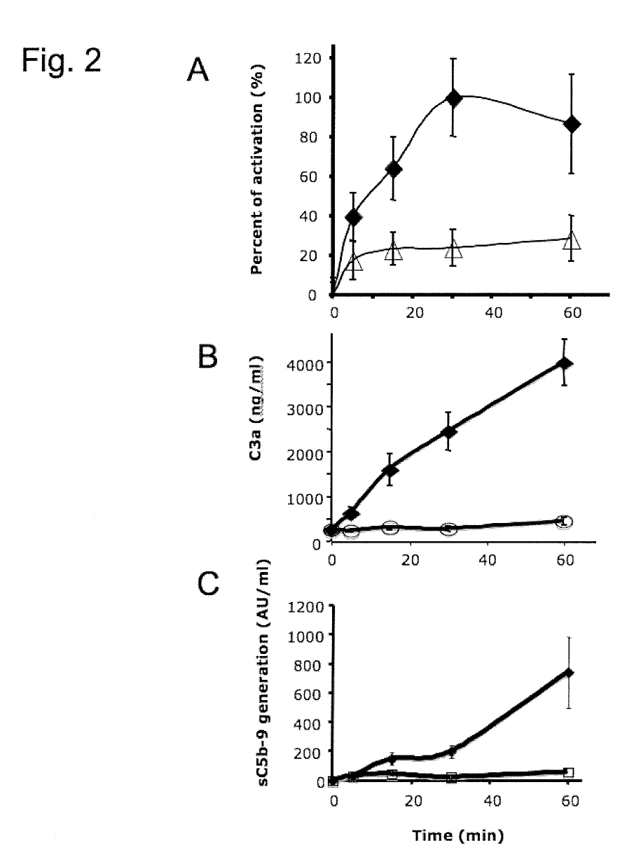

Complement activation in the presence and absence of compstatin: For kinetic analyses, islets were incubated in AB0-compatible plasma for 0, 5, 15, or 60 min in the presence or absence of the complement inhibitor compstatin (10 μM), then analyzed by COPAS (FIGS. 2A-2C). In the absence of compstatin, C3b/iC3b fragments were detected on the islets after as little as 5 minutes, and the binding of C3b/iC3b continued to increase over time. Addition of compstatin significantly reduced the binding of C3b/iC3b to the islets (FIG. 2A).

Assessment of C3a and sC5b-9 in the supernatants of the cultured islets confirmed this observation (FIGS. 2B and 2C). In contrast to the effect of compstatin, incubation of the islets in whole blood anticoagulated with hirudin had no effect on the generation of C3a and sC5b-9 (n=4).

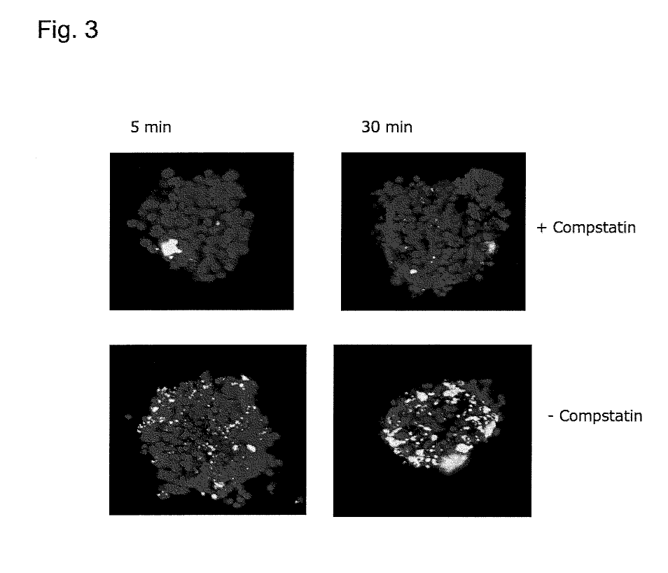

Examination of plasma-incubated islets by confocal microscopy confirmed that binding of C3b/iC3b had already occurred after 5 minutes, and it further demonstrated that after 30 minutes, the islets were covered by C3b/iC3b (FIG. 3). In contrast, addition of compstatin to the islets totally prevented C3b/iC3b binding at both 5 and 30 minutes.

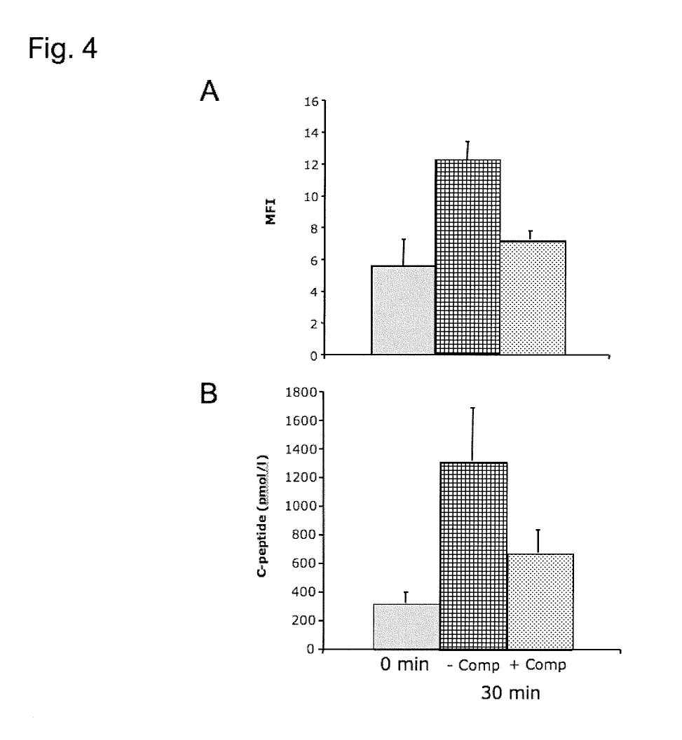

Lysis of islet cells as a result of complement attack: In order to determine whether complement activation led to lysis of islet cells, the islets were incubated in AB0-compatible plasma at 37° C. and stained with PI (FIG. 4A). COPAS analysis showed a significant increase in PI staining after 30 minutes. This staining was totally abrogated in the presence of 10 μM compstatin. After incubation of the islets at 37° C. for 24 hours, no binding of annexin V was detected.

Similar results were obtained when the supernatants were analyzed for C-peptide as a marker for islet destruction (FIG. 4B). A significant release of C-peptide was found after incubation of the islets for 30 minutes at 37° C. This release was totally inhibited in the presence of 10 μM compstatin.

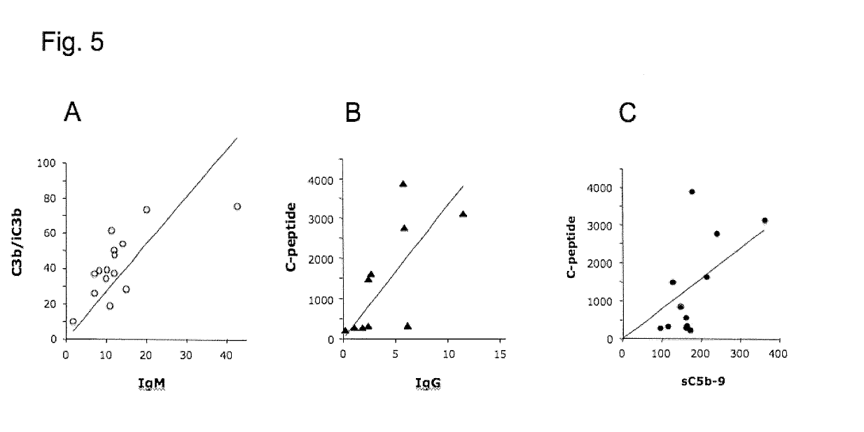

Correlation between binding of IgM, complement activation, and release of C-peptide: Significant correlations were found between the binding of IgM and C3b/iC3b (rho=0.67; p=0.01), between IgG and C-peptide release (rho=0.83; p=0.01), and between sC5b-9 and C-peptide release (rho=0.62; p=0.04) for islets incubated in hirudin-treated AB0-compatible plasma for 30 minutes (FIG. 5).

Summary:

The presence of IgG, IgM, Clq, C4, C9 and MBL on intact pancreatic islets incubated in AB0-compatible plasma was demonstrated by COPAS. IgG, IgM, C4, C3 and C9 gave a strong signal in confocal microscopy analysis. Deposition of C3b/iC3b was observed within 5 minutes and after 30 minutes in plasma, the islets were more or less covered with C3b/iC3b. It is likely the polyclonal anti-C3c antibody used in these studies underestimated the amount of C3b/iC3b, since C3c was cleaved from the complex after approximately 20 minutes. The generation of fluid-phase complement products C3a and sC5b-9 supports the conclusion that the deposition of complement fragments on the islet surface is the result of complement activation. The conclusion is further supported by the observation that complement inhibitor compstatin abolished the deposition of C3b/iC3b on the surface and abrogated the generation of fluid-phase complement activation products.

These results demonstrate that complement triggered by antibodies and the classical pathway is an important player in damaging pancreatic islet cells in transplantation. Thus, inhibition of complement activation during pancreatic islet cell transplantation is expected to reduce or prevent the damage islet cell grafts during transplantation.

### Example 2

### Materials and Methods

Animals: Retired breeder pigs, weighing approximately 200 kg, were used as donors for all experiments. Cynomolgus monkeys (Macaca fascicularis; 3-6 years old; 4-6 kg) were used as recipients. All procedures using pigs were approved by the Swedish Council on Medical Ethics. Cynomolgus monkeys were housed according to the guidelines of the Belgian Ministry of Agriculture and Animal Care. All procedures using monkeys were approved by the local Ethical Committee for Animal Care of the Universite Catholique de Louvain.

Reagents: Low molecular weight dextran sulfate (LMW-DS) was purchased from Sigma Chemicals (MW 5000; St. Louis, Mo., USA). Heparin was purchased from LEO (5000 U/mL; Lowen, Sweden).

Blood samples: All blood samples from monkeys were drawn from a femoral vein catheter at 0, 20, and 40 min and at 1, 3 and 24 h after transplantation. Blood was drawn from healthy human blood donors into 7-mL tubes containing 500 pg of hirudin, a specific inhibitor of thrombin (Refludan; Pharmion Ltd, Cambridge, UK). To obtain plasma, the samples were centrifuged at 4500×g for 5 min. If not immediately analyzed for activated partial thromboplastin time (APTT), the samples were stored at −70° C.

Islet isolation: Isolation of porcine islets was performed as previously described (Brandhorst et al., (1999) Transplantation 68 (3): 355), with minimal modifications. The digestion temperature was maintained at 30-37° C. during the recirculation and dilution phases. Purified islet fractions were pooled and cultured at 37° C. in a humidified atmosphere with 5% CO₂ in CMRL 1066 medium (Biochrom, Berlin, Germany) supplemented with 20% porcine serum, 2 mM N-acetyl-L-alanyl-L-glutamine, 10 mM HEPES, 100 IU/ml penicillin, 100 μg/ml streptomycin (Biochrom), and 20 μg/mlciprofloxacin (Bayer, Leverkusen, Germany).

Evaluation of porcine islet quality: The in vitro function and viability of the porcine islets were assessed after overnight culture as described above. The following assays were performed (Brandhorst et al., (1999) Transplantation 68 (3): 355):

i. Viability: Islet viability was determined by trypan blue exclusion assay.

ii. Insulin release: Glucose-stimulated insulin release was measured during static incubation by an insulin enzyme immunoassay (DRG Instruments, Marburg, Germany) using human insulin as a standard. Insulin release was expressed in terms of the stimulation index, defined as the ratio of stimulated (16.5 mM glucose) to basal (1.65 mM glucose) insulin release.

iii. Insulin content: For assays of islet insulin content, 1-mL samples were washed with distilled water, then sonicated (Labsonic, Braun, Melsungen, Germany) for 30 seconds. A 200-μL aliquot of each sample was subjected to acid-ethanol extraction (0.18 M HCl) and used for insulin measurement. Another 100-μL aliquot was dried at 60° C. overnight for consecutive fluorometric DNA assays (Kissane et al., (1958) J Biol. Chem. 233 (1): 184), using calf thymus DNA type I (Sigma, Deisenhofen, Germany) as a standard.

iv. 24-h insulin secretion: Immediately after a medium change, 500-μL samples of the medium were taken in duplicate from the remaining Petri dishes for determination of insulin accumulation in the medium, in order to calculate the 24-hour insulin secretion by the islets.

v. Transplantation into nude mice: The in vivo function of the islets was assessed by transplanting aliquots of freshly isolated islet preparations (3,000 islet equivalents (IEQs)) under the kidney capsule of diabetic athymic mice (Mollegaard, Lille Skensved, Denmark). Diabetes was induced by a single intravenous (i.v.) injection of 240 mg/kg body weight streptozotocin (STZ; Sigma, Deisenhofen, Germany) 5 to 7 days before transplantation. Only mice with serum glucose levels >19 mmol/L were selected as recipients. Transplantation was performed after exposure of the kidney through a flank incision under methofane anesthesia (Janssen-Cilag, Neuss, Germany), with the islet aliquot being placed under the kidney capsule by longitudinal injection through the kidney parenchyma from the posterior surface to the opposite pole. Postprandial serum glucose levels and the recipient's body weight were measured daily during the first week after transplantation and every third day for the rest of the observation period. The function of the transplanted islets in diabetic recipient mice was defined in terms of the normalization of postprandial serum glucose (<11 mmol/L). Graft removal was performed to confirm the graft function by nephrectomy under methofane anesthesia.

Islet transplantation: Before each experiment, the monkeys were sedated with 6 mg/kg Zoletil® 100 (Virbac S. A., Carros, France) intramuscularly, and general anesthesia was maintained with inhalation of 1-3% enflurane. During the experiment, electrocardiogram, blood pressure, and pulse were continuously monitored. The pig islets were suspended in 10 mL of transplant medium (Ringer acetate; Braun, Melsungen, Germany) with 25% human albumin and 5 mM glucose and injected slowly into the portal vein over the course of 5 minutes. The animals were treated in pairs, with each pair being given porcine islets from the same donor. One recipient in each pair received LMW-DS (monkeys M-5, M-7, M-9) and the other heparin as a control (monkeys M-6, M-8, M-10):

i. LMW-DS: Intravenous infusion of LMW-DS was performed via an indwelling catheter placed in the jugular vein or via a catheter in the portal vein. In the LMW-DS-treated groups, Promiten (Pharmalink AB, Upplands Vasby, Sweden) was injected i.v. just before islet transplantations to avoid the risk of anaphylactoid reactions triggered by LMW-DS. After the injection of Promiten, the monkey received a bolus dose of LMW-DS (1.5 mg/kg) i.v. prior to islet infusion, followed by 3.0 mg/kg LMW-DS given together with the porcine islets (10,000 IEQs/kg of recipient BW). The transplantation was followed by a continuous i.v. infusion of LMW-DS (1.0-1.5 mg/kg/h) for up to 24 h.

ii. Heparin: In the heparin-treated groups, the monkeys received a continuous i.v. infusion of heparin (35 U/kg of BW, heparin LEO, 5000 U/mL; Lowen, Sweden) for 24 hours, beginning immediately prior to islet infusion.

Analyses of blood and plasma samples: Activated Partial Thromboplastin Time (APTT) measurements were performed as previously described (Johansson et al., (2006) Am J. Transplant. 6 (2): 305). In order to compare actual blood concentrations of LMW-DS, the APTT value for each individual was converted to an LMW-DS concentration (mg/L), by adding known amounts of LMW-DS to the individual (and corresponding) serum samples in vitro, then measuring the APTT for each concentration. The standard curve obtained was used to convert the APTT values for the monkeys to actual LMW-DS concentrations.

Platelet counts and differential leukocyte counts were obtained using a Coulter ACT-diff analyzer (Beckman Coulter, Miami, Fla.) and EDTA-treated blood. Plasma levels of thrombin-antithrombin (TAT) were quantified using commercially available EIA kits (TAT, Behringswerke, Marburg, Germany). C3a generation was measured in plasma according to the method of Nilsson Ekdahl et al. ((1992) Scand J. Immunol. 35 (1): 85), and sC5b-9 was analyzed using a modification of the enzyme immunoassay described by Nilsson Ekdahl et al. ((1992) Scand J. Immunol. 35 (1): 85) and Mollnes et al. ((1995) Artif Organs 19 (9): 909).

Plasma IL-6, TNFα, IL-1β, and CRP were measured using a commercial ELISA kit (Immulite IL-6, Immulite TNFα, Immulite IL-1β, and Immulite High Sensitivity CRP, respectively; Diagnostic Products Corporation, Los Angeles, Calif., USA).

Histological and immunohistochemical staining: The monkey livers bearing transplanted adult porcine islet grafts were retrieved 24 hours after transplantation, at a time when the major part of the IBMIR has generally occurred (Bennet et al., (2000) Transplantation 69 (5): 711). Some tissue samples were snap-frozen in isopentane and stored at −70° C. Other samples were fixed with 4% paraformaldehyde overnight, then embedded in paraffin. The samples were sectioned and subsequently used for morphological scoring after hematoxylin eosin staining.

Immunohistochemical staining was carried out using guinea pig anti-insulin (DAKO, Carpenteria, Calif., USA), mouse anti-human neutrophil elastase (DAKO), mouse anti-human CD68 (DAKO), mouse anti-human MAC387 (Abcam, Cambridge, UK), mouse anti-human CD56 (Monosan, Stockholm, Sweden), rabbit anti-human CD3 (DAKO), mouse anti-human CD20 (DAKO), rabbit anti-human IgG and IgM (DAKO), mouse anti-human CD41 (DAKO), mouse anti-human C3c (QUIDEL, San Diego, Calif., USA), or goat anti-human C9 (Serotec Ltd Scandinavia, Oslo, Norway).

Treatment of porcine islets with human plasma: Approximately 1000 pig islets/40 μL of plasma (typically 5000 islets in 200 μL) were incubated in human hirudin-treated plasma in heparinized test tubes. Five different islet preparations and five different plasma preparations were used in these experiments. In some experiments, hirudin-treated plasma was preincubated with 10 μM (final concentration) of the potent compstatin analog, Ac-ICV(1MeW)QDWGAHRCT-NH₂ (SEQ ID NO: 4; Katragadda et al., (2006) J Med. Chem. 49 (15): 4616), for 15 minutes at 37° C. before the islets were added. The mixture of islets and plasma was then incubated, with gentle shaking, at 37° C. for up to 30 min. After centrifugation, the islets were immediately prepared for COPAS analysis and confocal microscopy.

Preparation of islets for flow cytometry and confocal microscopy: Ten microliters of FITC-labeled antibody recognizing one of the following proteins was added to 5000 islets (corresponding to approximately 10×10⁶ cells) in 100 μL of PBS according to the manufacturer's recommendations for single cells: Clq (1.0 g/L; AbCam, Cambridge, UK); C3c (3.2 g/L, for detection of C3b and iC3b; DakoCytomation, Glostrup, Denmark); C4 (1.3 g/L; Dako); C9 (2.6 g/L; Dako); mannose-binding lectin (MBL) (0.7 g/L; Dako); IgG (2.6 g/L; Dako); or IgM (4.0 g/L; Dako). Irrelevant mouse IgG1 (0.1 g/L; Dako) was used as a negative control.

For all immunostaining experiments, the islets were incubated, while gently rotating on ice, for 30 minutes in the presence of an individual antibody. After being washed with PBS, the islets were treated with 1% formaldehyde (Apoteket, Gothenburg, Sweden) and kept on ice until analyzed.

Complex object parametric analyzer and sorter (COPAS) analysis: The fluorescence-stained islets were analyzed using a complex object parametric analyzer and sorter (COPAS; Union Biometrica, Somerville, Mass.), which is a large particle-based flow cytometry instrument previously used for islet analysis (Fernandez et al., (2005) Transplantation 80 (6): 729). For each experiment, 1000 islets were collected using a 488/514 multi-line laser, and positive cells were sorted out for further analysis by confocal microscopy. The COPAS flow cytometry data were analyzed using CellQuest™ Pro software (BD Biosciences Immunocytometry Systems, San Jose, Calif.). Data were reported as mean fluorescent intensity (MFI).

Confocal microscopy: One to two hundred hand-picked, stained islets were contained in a drop of PBS in a small Petri dish and protected from light before examination in the confocal microscope (Zeiss 510 Meta confocal, Carl Zeiss, Jena, Germany). Examination of the stained islets was performed using the 488-nm laser at 10× magnification. Counterstaining with 4′,6-diamidino-2-phenylindole (DAPI) was used to visualize the nuclei of living islet cells.

Complement inhibition assay: One hundred microliters of 10% human serum (v/v), diluted in veronal buffer with 1 mM Ca²⁺, 0.3 mM Mg²⁺, 1% (w/v) BSA, and 0.05% (v/v) Tween 20, was incubated in the presence of serially diluted LMW-DS and/or compstatin in wells of microtiter plates for 30 minutes at 37° C. The wells were then washed with PBS containing 0.05% (v/v) Tween 20, and the bound C3 fragments were detected using 1004, of HRP-conjugated anti-C3c (Dako AS, Glostrup, Denmark).

Statistical analysis: All values are expressed as means±standard error of mean and were compared using Student's unpaired t-test or using the Wilcoxon rank-sum test. P values less than 0.05 were considered statistically significant.

### Experimental Example 5

### Islet Quality

The viability of the APIs used in this study was 96, 100, and 97%, respectively. The stimulation index in the SGS test was 1.29, 1.84, and 1.40, and the mean insulin content was 613, 149, and 685 μU/IEQs, respectively. Adult porcine islets used in each experiment cured diabetic athymic mice. When the possible detrimental effect of LMW-DS was assessed by incubating APIs from three different pancreata in the presence (100, 1000, or 2500 mg/L) or absence of LMW-DS, no adverse effect of LMW-DS on insulin release was observed at any of the concentrations tested. One of the transplanted monkeys (M6) treated with heparin died 2 hours after transplantation due to severe hypoglycemia.

Results:

Influence of LMW-DS on blood cell counts, liver and renal function, and cytokine induction in the transplanted monkeys. The platelet count and the creatinine levels were kept within normal ranges throughout the experiments. There was no difference in the liver enzymes at 24 hours after islet transplantation (AST, 434.7±126.4 vs. 288.0±130.4; ALT, 207.7±68.7 vs. 116.8±47.7). No bleedings or other adverse reactions were observed.

Influence of LMW-DS on cytokine induction was examined using three healthy monkeys. Only a slight increase in the IL-6 levels was seen 24 hours after administration of LMW-DS in two out of three healthy monkeys (maximum 27 μg/L). However, LMW-DS did not trigger an increase of plasma IL-1β, TNFα, or CRP.

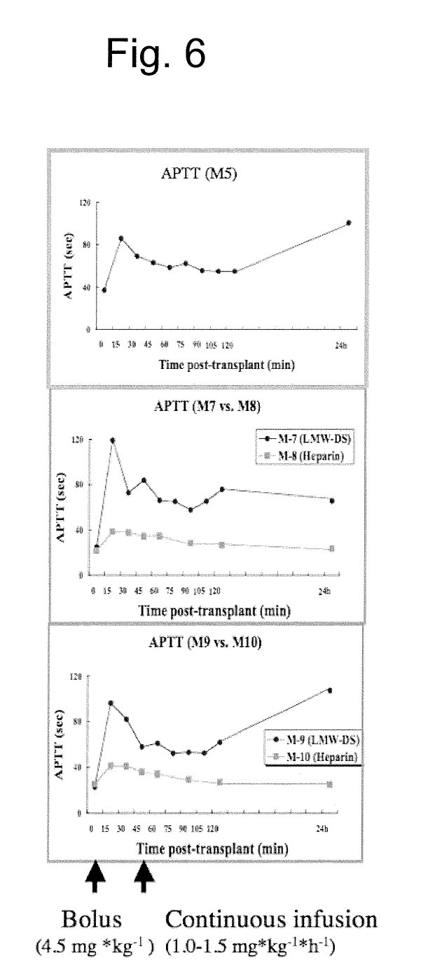

LMW-DS concentrations in transplanted monkeys. Previous studies showed a strong correlation between APTT and the concentration of LMW-DS (Johansson et al., (2006) Am J. Transplant. 6 (2): 305). Plasma APTT was therefore used to follow the blood concentration of LMW-DS in the transplanted monkeys (FIG. 6). The APTT in monkeys treated with heparin at concentrations routinely used in clinical islet transplantation (i.e., 500-1000 IU/L) was kept constant at 25-40 seconds (s) throughout the whole study period. The APTT in monkeys treated with LMW-DS reached around 100s at 15 minutes after islet infusion, but gradually decreased during 2 hours after islet transplantation. After 24 hours, the APTTs in monkeys M-5, M-7, and M-9 were 101s, 66s, and 107s, respectively. After this conversion, the APTTs at 24 hours corresponded to a plasma concentration of LMW-DS of 30 mg/L (101s); corresponded to 15 mg/L (66s); and 35 mg/L (107s), respectively. Thus, both M-5 and M-9 had more than double concentration of LMW-DS compared to M-7.

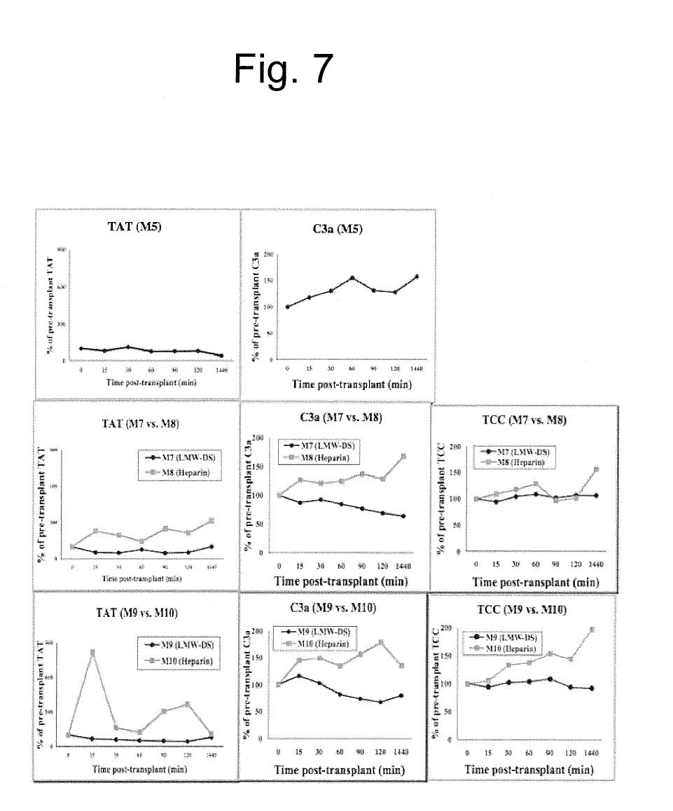

Inhibition of the IBMIR by LMW-DS during pig islet xenotransplantation. LMW-DS, unlike heparin, diminished both the coagulation and the complement cascade activation in two sets of monkeys. The increase of coagulation marker TAT was effectively inhibited by LMW-DS (FIG. 7). The complement activation parameters C3a and sC5b-9 were also suppressed by LMW-DS in both treated monkeys compared to the controls during the study period (FIG. 7). In M-5, TAT was totally suppressed while C3a was more difficult to evaluate without the corresponding control (M6). In this animal, sC5b-9 was not assessed due to an insufficient amount of plasma samples.

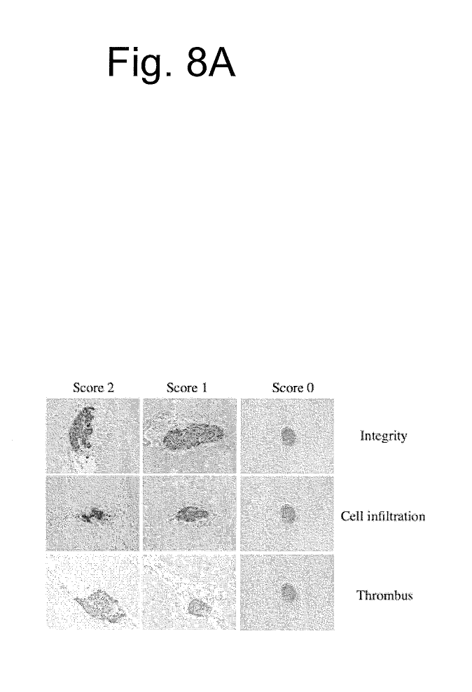

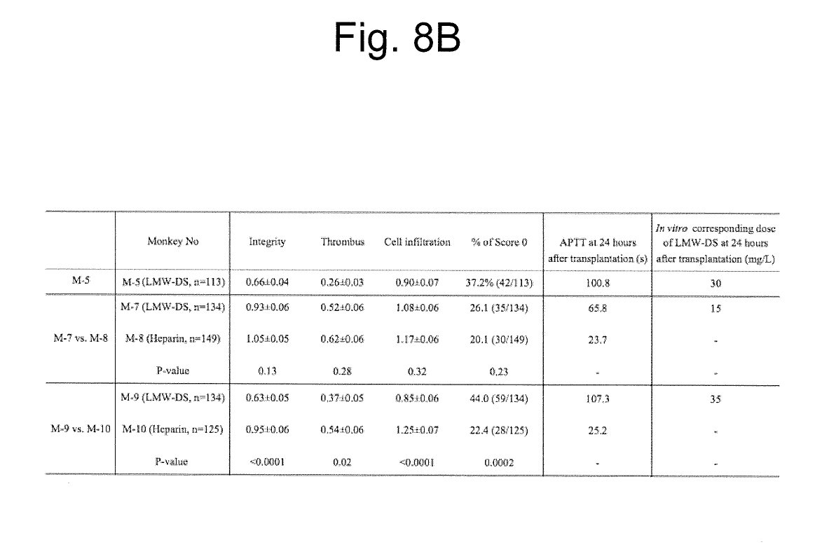

Histological evaluation of grafted pig islets after intraportal transplantation into the monkeys treated with LMW-DS or heparin. Morphological aspects of islet grafts were scored semiquantitatively according to the representative examples shown in FIG. 8. As summarized in the figure, histology of the transplanted grafts were well kept in the monkeys treated with LMW-DS in both settings of experiments. However, the beneficial effects of LMW-DS were more pronounced in M-6 and M-9 compared to M-7. Indeed, the completely preserved islets (score 0 in all categories) were encountered in 37.2% and 44% of the LMW-DS treated animals M-6 and M-9 (LMW-DS treated monkeys), respectively, but in only 22% of the control M-10.

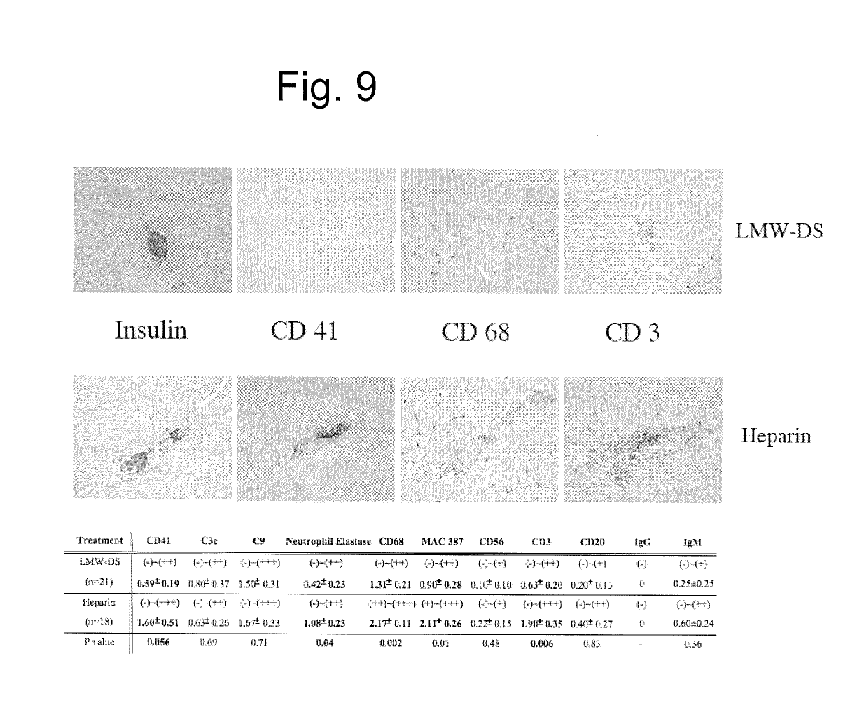

Immunohistochemical staining of grafted pig islets after intraportal transplantation into the monkeys treated with LMW-DS or heparin. The immunohistochemical findings from the grafts were summarized in FIG. 9. As expected, most parameters involved in innate immune responses were active after 24 hours post islet transplantation in the controls M8 and M10. In particular, CD41⁺ platelets, CD68⁺ macrophages, and neutrophil elastase positive PMNs were abrogated in the monkeys treated with LMW-DS compared to the controls given heparin (p=0.056, 0.002, and 0.04, respectively), but CD56⁺ NK cells were found only occasionally. Unlike the soluble complement markers, there was no clear inhibition of complement activation as reflected in deposition of C3 fragment and C9 on the surface of the islets. Furthermore, IgM antibodies were found on islet both in LMW-DS and heparin-treated animals. Most of parameters reflecting specific immune responses were yet silent. However, CD3+ T cell infiltration was already observed in the islet grafts of the controls M8 and M10. Notably, this infiltration was effectively suppressed by LMW-DS.

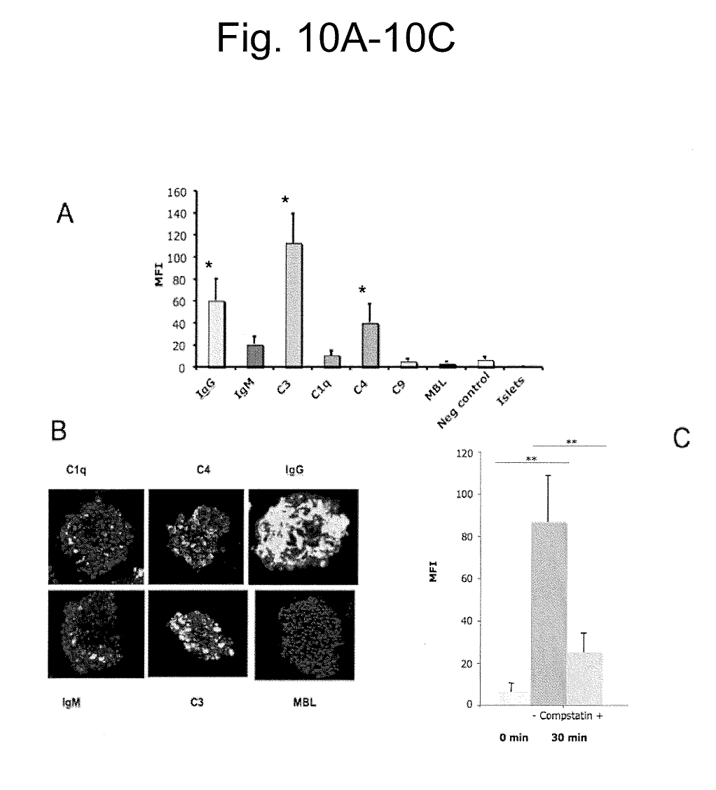

Binding of complement components to porcine islets after incubation in human plasma. After incubation in hirudin-treated plasma, the porcine islets were stained with FITC-conjugated antibodies recognizing IgG, IgM, Clq, C3b/iC3b, C4 fragments, C9, CRP, and MBL. Large particle flow cytometry and confocal microscopy demonstrated that antibodies against IgG, IgM, Clq, C4, and C3 bound strongly to the islets, but the binding of MBL and C9 was less prominent (FIGS. 10A and 10B). C3b/iC3b fragments were detected on the islets after only 5 minutes, and the binding of C3b/iC3b continued to increase over time. Addition of compstatin significantly reduced the binding of C3b/iC3b to the islets (FIG. 10C). Confocal microscopy analyses confirmed these results.

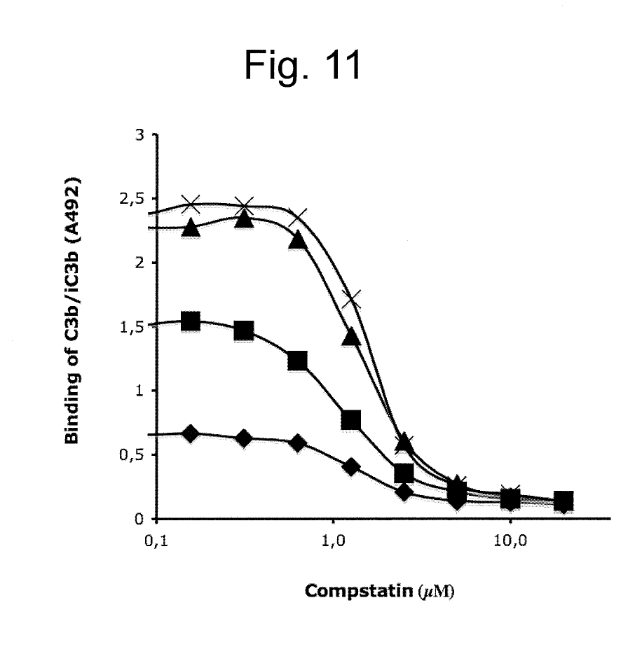

Inhibition of complement activation by LMW-DS and compstatin. 10% (v/v) human serum was incubated in wells of microtiter plates in the presence of LMW-DS and/or compstatin for 30 minutes at 37° C. (FIG. 11). In the absence of compstatin, LMW-DS inhibited complement activation only marginally between 10 and 100 mg/L and this inhibition was more pronounced at concentrations above this level. In the presence of compstatin, there was no effect below 0.5 μM of the compound, but at higher concentrations compstatin gradually inhibited complement activation. At 5 μM, total inhibition was achieved. There was no indication of interaction between the drugs regarding this effect on complement activation in serum.

Summary:

The xenogeneic IBMIR in the non-human primate model used in the experiments is characterized by an immediate binding of antibodies that triggers deleterious complement activation, and a subsequent clotting reaction that leads to further complement activation. Most parameters reflecting the IBMIR, i.e., both the coagulation and complement cascades, platelet deposition, and infiltration of macrophages and neutrophils, were attenuated in the monkeys treated with LMW-DS compared to the controls. Notably T cell infiltration observed in some of the transplanted islet grafts, was also effectively suppressed, demonstrating that the innate immune responses are attenuated by LMW-DS. LMW-DS and compstatin are effective in inhibiting the IBMIR. The data show that LMS-DS and compstatin do not interact in human serum.

These results demonstrate that immunoglobulin-triggered complement activation is an important player in IBMIR and the destruction of transplanted pancreatic islet cells. Thus, inhibition of complement activation and IBMIR during pancreatic islet cell transplantation is expected to reduce or prevent the damage islet cell grafts during transplantation.

The present invention is not limited to the embodiments described and exemplified above, but is capable of variation and modification within the scope of the appended claims.

What is claimed:

1. A method for reducing rejection of pancreatic islet cells transplanted into a subject, the method comprising transplanting pancreatic islet cells into a subject in the presence of a complement inhibitor, wherein the complement inhibitor inhibits complement activation, thereby reducing rejection of the transplanted pancreatic islet cells.

2. The method of claim 1, wherein the complement inhibitor inhibits release of C-peptide by the transplanted pancreatic islet cells.

3. The method of claim 1, wherein the complement inhibitor inhibits lysis of the transplanted pancreatic islet cells.

4. The method of claim 1, wherein the complement inhibitor is a C3 inhibitor.

5. The method of claim 4, wherein the C3 inhibitor is compstatin, a compstatin analog, a compstatin peptidomimetic, a compstatin derivative, or any combination thereof.

6. The method of claim 1, wherein the complement inhibitor is a MAC inhibitor.

7. The method of claim 6, wherein the MAC inhibitor is Eculizumab, Pexelizumab, ARC 1905 or any combination thereof.

8. The method of claim 1, wherein the transplanted pancreatic islet cells are allogeneic.

9. The method of claim 1, wherein the transplanted pancreatic islet cells are xenogeneic.

10. The method of claim 1, wherein the transplanted cells have increased engraftment compared to cells transplanted in the absence of the complement inhibitor.

11. The method of claim 1, wherein the subject is human.

12. The method of claim 1, wherein the subject has type 1 diabetes.

13. A method for reducing rejection of allogeneic or xenogeneic pancreatic islet cells transplanted into a subject, the method comprising transplanting allogeneic or xenogeneic pancreatic islet cells into a subject in the presence of a complement inhibitor and dextran sulfate, thereby reducing rejection of the transplanted pancreatic islet cells.

14. The method of claim 13, wherein the complement inhibitor is a C3 inhibitor.

15. The method of claim 14, wherein the C3 inhibitor is compstatin, a compstatin analog, a compstatin peptidomimetic, a compstatin derivative, or any combination thereof.

16. The method of claim 13, wherein the dextran sulfate is low molecular weight dextran sulfate.

17. The method of claim 13, wherein the transplanting step results in the inhibition of instant blood-mediated inflammatory reaction.

18. The method of claim 17, wherein inhibition of instant blood-mediated inflammatory reaction results in inhibition or prevention of at least one of: a decrease in free circulating platelets; infiltration of the transplanted pancreatic islet cells by polymorphonuclear lymphocytes; infiltration of the transplanted pancreatic islet cells by macrophages; infiltration of the transplanted pancreatic islet cells by neutrophils; and an increase in factor XIa-antithrombin (FXIa-AT), factor XIIa-antithrombin (FXIIa-AT), thrombin-antithrombin (TAT) and/or plasmin-alpha 2 antiplasmin (PAP).
